(19) United States
(12) Patent Application Publication
(10) Pub. No.: US 2020/0058141 A1
(43) Pub. Date: Feb. 20, 2020

(54) IMAGE CAPTURE AND RECONSTRUCTION PROTOCOL SELECTION SYSTEM

(71) Applicant: CARESTREAM HEALTH, INC., Rochester, NY (US)

(72) Inventors: Richard A. SIMON, Rochester, NY (US); Lawrence A. RAY, Rochester, NY (US); Edward B. GINDELE, Rochester, NY (US); Nathan J. PACKARD, Provo, UT (US)

(21) Appl. No.: 16/102,967

(22) Filed: Aug. 14, 2018

(51) Int. Cl. G06T 11/00; A61B 6/00; A61B 6/03

(52) U.S. Cl. CPC G06T 11/005; A61B 6/4085; A61B 6/032; G06T 2211/424; A61B 6/466; A61B 6/5294; G06T 2211/408; A61B 6/463

(57) ABSTRACT

A method accepts patient-specific data for imaging a patient extremity on a cone beam computed tomography apparatus and generates and displays a set of acquisition parameters and dose indication for 2-D projection image capture of the extremity. The generated set of acquisition parameters and dose indication are updated according to one or more operator entered instructions. A set of 2-D projection images of a 3-D volume is acquired according to the updated set of acquisition parameters. The volume is reconstructed according to the acquired set of 2-D projection images.

### FIELD OF THE INVENTION

The disclosure relates generally to diagnostic imaging and in particular to cone beam imaging systems that have a selectable arrangement of x-ray sources for obtaining volume images of localized features and extremities.

### BACKGROUND

3-D volume imaging has proved to be a valuable diagnostic tool that offers significant advantages over earlier 2-D radiographic imaging techniques for evaluating the condition of internal structures and organs, 3-D imaging of a patient or other subject has been made possible by a number of advancements, including the development of high-speed imaging detectors, such as digital radiography (DR) detectors that enable multiple images to be acquired in rapid succession.

Cone beam computed tomography (CBCT) or cone beam CT technology offers considerable promise as one type of diagnostic tool for providing 3-D volume images. Cone beam CT systems capture volumetric data sets by using a high frame rate digital radiography (DR) detector and an x-ray source, typically affixed to a gantry that rotates about the object to be imaged, directing, from each of a set of relative angular positions in its orbit around the subject, a divergent cone beam of x-rays toward the subject. The CBCT system captures projections at regular angular increments during its orbit about the subject, for example, one 2-D projection image at every degree of rotation. The projections are then reconstructed into a 3D volume image using various techniques. Among well known methods for reconstructing the 3-D volume image from the 2-D image data are filtered back projection approaches.

CBCT apparatus have considerable promise in orthopedic imaging and for related, localized imaging of extremities, such as hands, arms, legs, ankles, and knees. Portable and smaller-scale CBCT systems have been designed to allow imaging of various extremities under various conditions, including imaging of limbs in weight-bearing or elevated positions, for example. One feature of advanced CBCT systems includes configuration with multiple x-ray sources, offering the practitioner the capability to select a particular x-ray source with suitable attributes for an exam, or to utilize multiple x-ray sources in a particular imaging session.

Orthopedic applications introduce a broad range of variability, even where the same anatomy features are the subject of the CBCT imaging exam. For any particular patient, there can be a number of factors that influence the decision-making process for selecting suitable radiation conditions. The size, condition, behavior, and age of the patient, for example, may dictate the use of one type of x-ray source or another, or the use of multiple sources for enhanced resolution, for example, or may dictate the need for reduced exam timing. The particular condition of interest, required patient posture for extremity positioning or stress applied to a limb or other extremity, and other aspects of the exam can all play a role in source selection and in determining suitable technique and related parameters. In addition to number and type of x-ray sources, other features of the CBCT system itself can also be of interest, including spectral range, filter selection, number of projection images, and collimation settings, for example.

Thus, unlike many situations with conventional 2D radiography exam types, there can be a sizable number of factors to be considered in CBCT imaging, particularly for extremities. It can be appreciated that there would be value in providing utilities that streamline the decision process and thus improve workflow for CBCT imaging.

### SUMMARY

An aspect of this application is to advance the art of medical digital radiography, particularly for 3D volume imaging.

Another aspect of this application is to address, in whole or in part, at least the foregoing and other deficiencies in the related art.

It is another aspect of this application to provide, in whole or in part, at least the advantages described herein.

According to an embodiment of the present disclosure, there is provided a method comprising: a) accepting patient-specific data for imaging a patient extremity on a cone beam computed tomography apparatus; b) generating and displaying, according to the patient-specific data, a set of acquisition parameters and a dose indication for 2-D projection image capture of the extremity on the cone beam computed tomography apparatus; d) updating the generated set of acquisition parameters and dose indication according to one or more operator-entered instructions; e) acquiring a set of 2-D projection images of a 3-D volume that includes the patient extremity according to the updated set of acquisition parameters; f) reconstructing the 3-D volume according to the acquired set of 2-D projection images; and g) displaying, storing, or transmitting the reconstructed 3-D volume.

These objects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a schematic diagram that shows the circular scan paths for a radiation source and detector when imaging the right knee.

FIG. 2 shows a top and perspective view of the scanning pattern for an imaging apparatus according to an embodiment of the application.

FIG. 3A shows a perspective view of patient access to a CBCT imaging apparatus.

FIG. 3B shows a top view of patient access to a CBCT imaging apparatus.

FIG. 3C shows a perspective view of the apparatus with transport mechanisms oriented to allow the patient to be in seated position for imaging.

FIG. 4 show portions of the operational sequence for obtaining CBCT projections of a portion of a patient's extremity at a number of angular positions.

FIG. 5 is a perspective view showing the apparatus in position for patient imaging of the knee.

FIG. 6 is a perspective view that shows a CBCT imaging embodiment that has multiple radiation sources.

FIG. 7 is a logic flow diagram that shows a sequence for automated parameters setup according to an embodiment of the present disclosure.

FIG. 8 shows a display screen that can appear on control panel 124 for showing various parameters related to a CBCT exam for an extremity.

### DESCRIPTION OF EXEMPLARY EMBODIMENTS

The following is a description of exemplary embodiments of the invention, examples of which are illustrated in the accompanying drawings. Wherever possible, the same reference numbers will be used throughout the drawings to refer to the same or like parts.

For illustrative purposes, principles of the invention are described herein by referring mainly to exemplary embodiments thereof. However, one of ordinary skill in the art would readily recognize that the same principles are equally applicable to, and can be implemented in, all types of radiographic imaging arrays, various types of radiographic imaging apparatus and/or methods for using the same and that any such variations do not depart from the true spirit and scope of the application. Moreover, in the following description, references are made to the accompanying figures, which illustrate specific exemplary embodiments. Electrical, mechanical, logical and structural changes can be made to the embodiments without departing from the spirit and scope of the invention.

In the context of the application, the term “extremity” has its meaning as conventionally understood in diagnostic imaging parlance, referring to knees, legs, ankles, fingers, hands, wrists, elbows, arms, and shoulders and any other anatomical extremity. The term “subject” is used to describe the extremity of the patient that is imaged, such as the “subject leg”, for example. The term “paired extremity” is used in general to refer to any anatomical extremity wherein normally two or more are present on the same patient. In the context of the application, the paired extremity is not imaged unless necessary; only the subject extremity is imaged. In one embodiment, a paired extremity is not imaged to reduce patient dose.

A number of the examples given herein for extemporary embodiments of the application focus on imaging of the load-bearing lower extremities of the human anatomy, such as the leg, the knee, the ankle, and the foot, for example. However, these examples are considered to be illustrative and non-limiting.

In the context of the application, the term “arc” or, alternately, or arcuate has a meaning of a portion of a curve, spline or non-linear path, for example as being a portion of a curve of less than or greater than 360 degrees.

The term “actuable” has its conventional meaning, relating to a device or component that is capable of effecting an action in response to a stimulus, such as in response to an electrical signal, for example.

As used herein, the term “energizable” relates to a device or set of components that perform an indicated function upon receiving power and, optionally, upon receiving an enabling signal.

CT and CBCT systems are known apparatus used for volume imaging in dental and medical applications. An exemplary medical cone beam CT (CBCT) imaging system is the Carestream OnSight3D Extremity System from Carestream Health, Rochester, N.Y. Reference is made to commonly assigned U.S. Pat. No. 8,348,506 (Yorkston) entitled “EXTREMITY IMAGING APPARATUS FOR CONE BEAM COMPUTED TOMOGRAPHY” and to U.S. Pat. No. 9,949,703 (DiRisio) entitled “EXTREMITY IMAGING APPARATUS”, both incorporated herein by reference in their entirety.

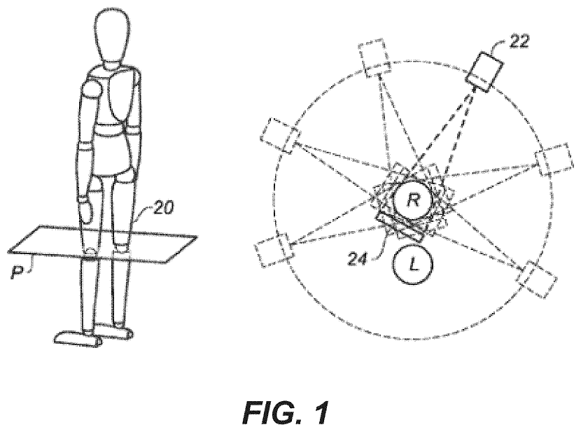

FIG. 1 illustrates CBCT imaging of a knee. The top view of FIG. 1 shows the circular scan paths for a radiation source 22 and detector 24 when imaging the right knee R of a patient as a subject 20. Various positions of radiation source 22 and detector 24 are shown in dashed line form. Source 22, placed at some distance from the knee, can be positioned at different points over an arc of about 200 degrees; with any larger arc the paired extremity, left knee L, blocks the way. Detector 24, smaller than source 22 and typically placed very near subject 20, can be positioned between the patient's right and left knees and is thus capable of positioning over the full circular orbit. The scan path of the source and/or detector may define a source and/or detector plane P through which the source and/or detector travel as they traverse respective source and detector paths around a region of the subject 20.

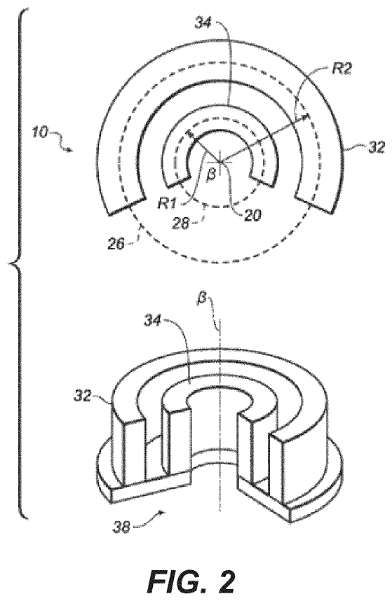

A full 360-degree orbit of the source and detector is not needed for conventional CBCT imaging; instead, sufficient information for image reconstruction can be obtained with an orbital scan range that just exceeds 180 degrees by the angle of the cone beam itself, for example. However, in some cases it can be difficult to obtain much more than about 180-degree revolution for imaging the knee or other joints. Moreover, there can be diagnostic situations in which obtaining projection images over a certain range of angles has advantages, but patient anatomy blocks the source, detector, or both from imaging over that range. Some of the proposed solutions for obtaining images of extremities under these conditions require the patient to assume a position that is awkward or uncomfortable. The position of the extremity, as imaged, is not representative of how the limb or other extremity serves the patient in movement or under weight-bearing conditions. It can be helpful, for example, to examine the condition of a knee or ankle joint under the normal weight load exerted on that joint by the patient as well as in a relaxed position. But, if the patient is required to assume a position that is not usually encountered in typical movement or posture, there may be excessive strain, or insufficient strain, or poorly directed strain or tension, on the joint. The knee or ankle joint, under some artificially applied load and at an angle not taken when standing, may not behave exactly as it does when bearing the patient's weight in a standing position. Images of extremities under these conditions may fail to accurately represent how an extremity or joint is used and may not provide sufficient information for assessment and treatment planning. The perspective and corresponding top view of FIG. 2 show how the scanning pattern is provided for components of CBCT imaging apparatus 10 according to one embodiment. A detector path 28, shaped as a circular arc of a suitable radius R1 from a central axis β is provided for a digital radiation detector by a detector transport mechanism 34. A source path 26 shaped as a circular arc of a second, larger radius R2 is provided for a radiation source by a source transport mechanism 32. In one embodiment, a non-linear source path 26 is greater in length than a non-linear detector path 28. According to an embodiment of the application, described in more detail subsequently, the same C-shaped transport system provides both detector transport 34 and source transport 32. The extremity of subject 20 is preferably substantially centered along central axis β so that central axis β can be considered as a line through points in the extremity of subject 20. In one embodiment, an imaging bore of the CBCT apparatus can include or encompass the central axis β.

The limiting geometry for image capture is due to the arc of source transport 32 blocked by patient anatomy, such as by a paired limb), and thus limited typically to less than about 220 degrees, as noted previously. The circumferential gap, or opening, or housing gap, or peripheral gap 38 exists between the endpoints of the arc of source path 26, between ends of a C-shaped transport mechanism, or between ends of a C-shaped scanner housing as described herein. Gap or opening 38 provides space for the patient to stand, for example, while one leg is being imaged. It also provides access through the gap for positioning a patient extremity by moving the patient extremity through the gap in a direction substantially perpendicular to the axis β without requiring the extremity extended through one end of the bore opening.

Detector path 28 can extend across housing gap 38 to allow scanning, since the detector is not necessarily blocked by patient anatomy and can have a detector travel path extending between a patient's extremities and at least partially around an imaged extremity. Embodiments of the present invention allow clearance of the housing gap by moving away a housing extension, or door, and scanning components, as explained herein, to allow access for the patient as part of initial patient positioning. The perspective view in FIG. 2, for example, shows detector transport 34 rotated to open up circumferential gap 38.

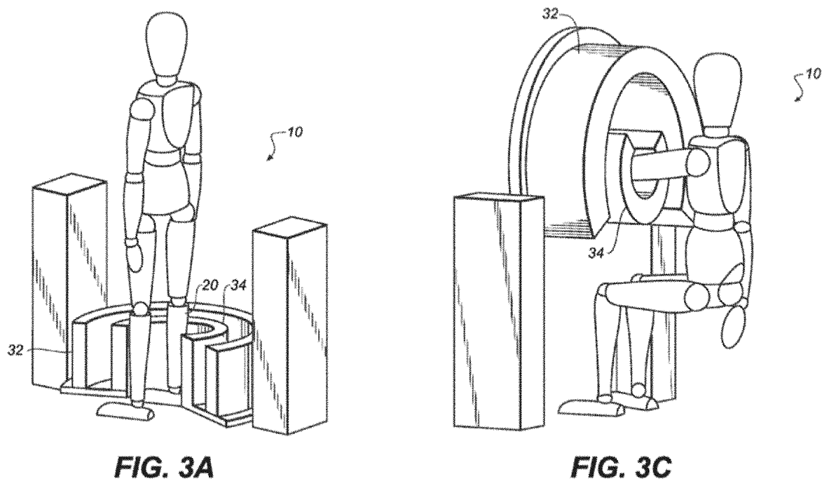

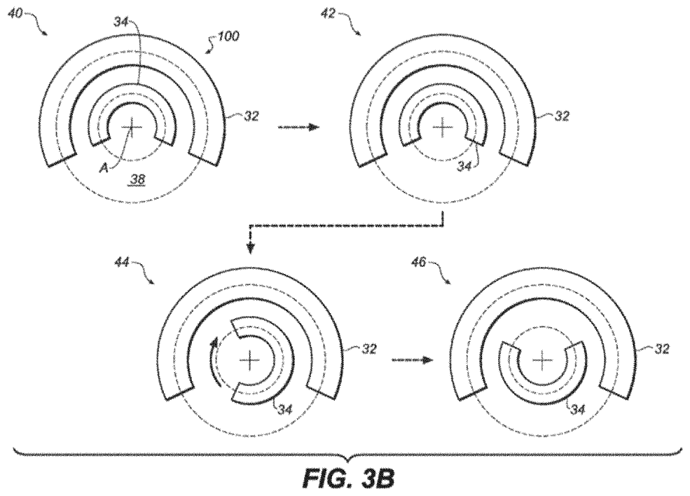

FIGS. 3A, 3B, and 3C show aspects of patient access, system operation, and positioning within CBCT imaging apparatus 10 according to an embodiment of the present disclosure. With reference to FIG. 3A, detector transport 34 may be translated to the open position, as shown in FIG. 3A, and the patient can freely move in and out of position for imaging with reference to an imaging axis A, corresponding to FIG. 2 axis β. When the patient is properly in position, detector transport 34 is revolved about axis β by more than 180 degrees; according to an embodiment of the invention, detector transport 34 is revolved about axis β by substantially 200 degrees.

This patient access and subsequent adjustment of detector transport 34 is shown in successive stages 42, 44, and 46 in FIG. 3B. This orbital movement confines the extremity to be imaged more effectively and places detector 24, not visible in FIGS. 2-3B due to the detector transport 34 housing, in position near subject 20 for obtaining the first projection image in sequence. In one embodiment described herein, a detector transport 34 can include shielding, a housing extension, a door, or a combination thereof, over part of the detector path, and/or the gap 38.

Circumferential gap or opening 38 not only allows access for positioning of a subject's leg or other extremity, but also allows sufficient space for the patient to stand in normal posture during imaging, placing the subject leg for imaging in the central position along axis β. (FIG. 2) and the non-imaged paired leg within the space defined by circumferential gap 38. Circumferential gap or opening 38 extends approximately 360 degrees minus the fan angle (e.g., between ends of the source path or ends of the C-shaped housing), which is determined by source-detector geometry and distance. Circumferential gap or opening 38 permits access for easily positioning the extremity so that it can be centered along central axis β. Once the patient's leg or other extremity is in place, detector transport 34, a hooded cover, hollow door, housing extension, or other member in the transport path, can be revolved into position, enclosing the detector path in the circumferential housing gap or opening 38. FIG. 3C shows a perspective view of the apparatus 10 having transport mechanisms 32 and 34 oriented to allow the patient to be in seated position for imaging.

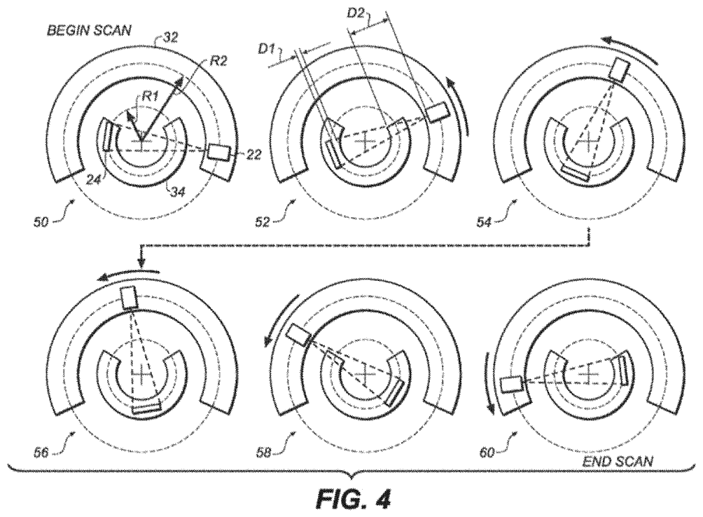

By way of example, the top views of FIG. 4 show portions of the operational sequence for obtaining CBCT projections of a portion of a patient's extremity at a number of angular positions when using the CBCT imaging apparatus 10. The relative positions of radiation source 22 and detector 24, which may be concealed under a hood or chassis, as noted earlier, are shown in FIG. 4. The source 22 and detector 24 can be aligned so the radiation source 22 can direct radiation toward the detector 24 (e.g., diametrically opposite in relation to axis β) at each position during the CBCT scan and projection imaging. The sequence begins at a begin scan position 50, with radiation source 22 and detector 24 at initial positions to obtain an image at a first angle. Then, both radiation source 22 and detector 24 revolve about axis β as represented in interim scan positions 52, 54, 56, and 58. Imaging terminates at an end scan position 60. As this sequence shows, source 22 and detector 24 are in opposing positions relative to axis β at each imaging angle. Throughout the scanning cycle, detector 24 is within a short distance D1 of subject 20. Source 22 is positioned at a longer distance D2 from subject 20. The positioning of source 22 and detector 24 components can be carried out by separate actuators, one for each transport path, or by a single rotatable member, as described in more detail subsequently. It should be noted that scanning motion in the opposite direction, that is, clockwise with respect to the example shown in FIG. 4, is also possible, with the corresponding changes in initial and terminal scan positions.

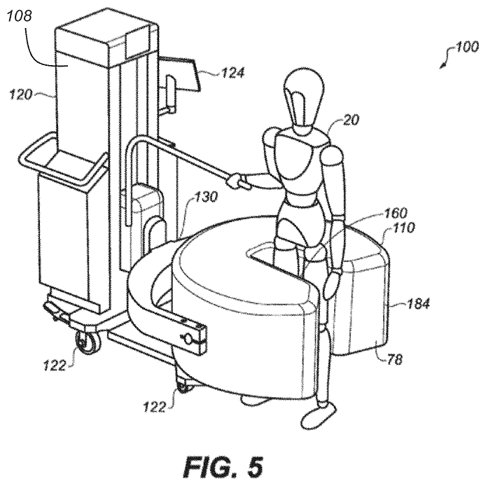

Given this basic operation sequence in which the source 22 and detector 24 orbit the subject extremity, the usefulness of an imaging system that is adaptable for imaging patient extremities with the patient sitting or standing and in load-bearing or non load-bearing postures can be appreciated. The perspective view of FIG. 5 shows a CBCT imaging apparatus 100 for extremity imaging according to an embodiment of the invention. Imaging apparatus 100 has a gimbaled imaging, or scanning, apparatus 110 with a housing 78 that conceals source 22 and detector 24 therewithin. Imaging apparatus, or scanner, 110 is adjustable in height and rotatable in gimbaled fashion about non-parallel axes, such as about substantially orthogonal axes, as described in subsequent figures, to adapt to various patient postures and extremity imaging conditions. A support column 120 supports scanner 110 on a yoke, or bifurcated or forked support arm 130, a rigid supporting element that has adjustable height and further provides rotation of scanner 110 as described subsequently. Support column 120 can be fixed in position, such as mounted to a floor, wall, or ceiling.

According to portable CBCT embodiments such as shown in FIG. 5, support column 120 mounts to a support base that also includes optional wheels or casters 122 for transporting and maneuvering imaging apparatus 100 into position within a C-shaped housing 184 having a door 160. A scanner housing 78 protects the source and detector components. A control panel 124 can provide an operator interface, such as a display monitor, for entering instructions to a control logic processor 108 that is in signal communication with control panel 124 for apparatus 100 adjustment and operation. In one embodiment, the control panel 124 can include a processor or computer (e.g., hardware, firmware and/or software) to control operations of the CBCT system 100. Support column 120 can be of fixed height or may have telescoping operation, such as for improved visibility when apparatus 100 is moved.

As the various embodiments shown in FIGS. 3A, 3C, and 5 indicate, the CBCT apparatus 100 can be used with the patient seated or standing, and in weight-bearing and supported-weight positions, and adapts to size and disposition of the patient. In addition to adjustment of the scan path geometry for patient condition and exam type. CBCT imaging apparatus 100 also provides various options for adapting a suitable radiation sequence. The CBCT imaging apparatus 100 can have various arrangements of radiation sources, configurable to support different types of orthopedic imaging.

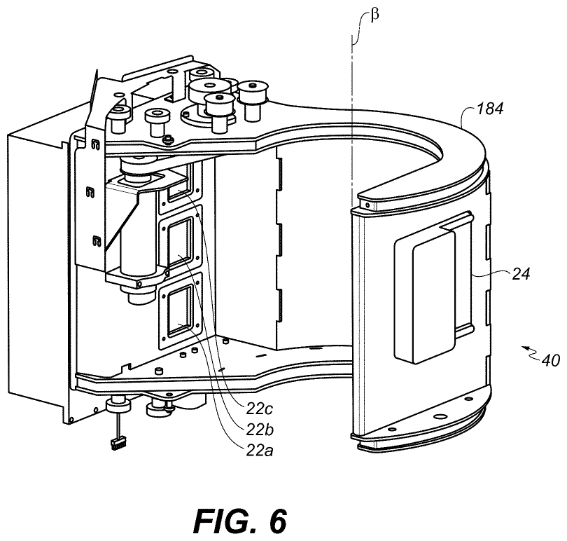

The perspective view of FIG. 6 shows a CBCT imaging embodiment in which housing 184 has a gantry 40 with multiple radiation sources 22a, 22b, and 22c. The different sources 22a, 22b, and 22c can be individually energized and can provide the same or different types of radiation emission. The need for multiple sources can be based on factors such as patient size, limb type and orientation, patient condition or attitude, type of tissue, presence of metal or very dense materials, and the like.

For example, examination for a body part of moderate size, such as a hand, requires only a small volume for scanning, so that sufficient images for reconstruction can be obtained using a single x-ray source. Conversely, an examination where limb dimensions extend over a significant length can be more compatible for imaging of the volume using multiple sources.

In some cases, there can be factors that conflict in some ways, such as for imaging that is most favorable with higher resolution, but where low dose should be used.

Recognizing the inherent complexities of orthopedic imaging and the need for more efficient workflow and a streamlined decision-making process, the Applicants have developed an approach for automated assistance with orthopedic imaging setup when using portable CBCT extremity imaging apparatus.

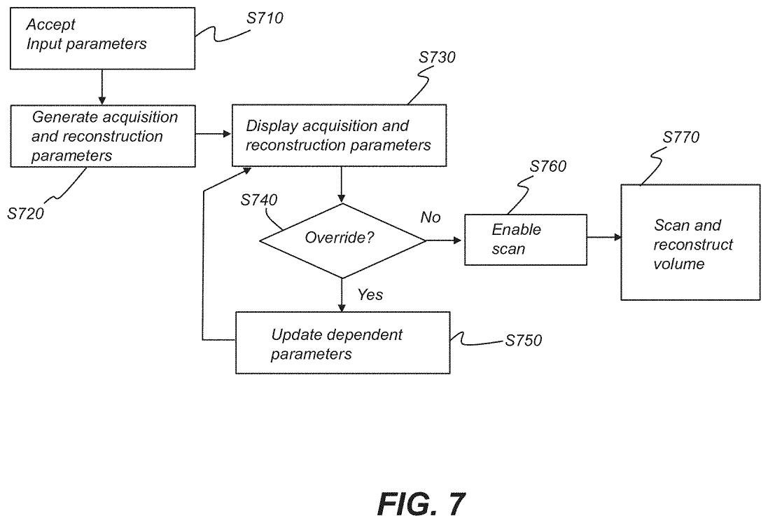

The logic flow diagram of FIG. 7 shows a sequence for automated parameters setup according to an embodiment of the present disclosure. The sequence can be executed, for example, on control logic processor 108, as shown in the FIG. 5 embodiment, with displayed parameters on control panel 124. In an accept parameters step S710, the system acquires information regarding an upcoming exam, with patient-specific data and clinical parameters such as those described in more detail subsequently. Input parameters can be provided from operator entry, from patient records or work order, or from a combination of sources that store and provide patient data.

The patient-specific data and clinical parameters acquired in step S710 provide a basis of information that guides selection logic for an automated parameters generation step S720, in which acquisition and reconstruction parameters suitable for the input parameters are generated. A display step S730 then shows the computed acquisition and reconstruction parameters that have been generated for the patient. Generating acquisition parameters can include selecting one set of parameters from a library having multiple sets, each with recommended parameter settings, previously generated and stored, for example. An override decision step S740 gives the operator or practitioner the option to review and accept or override the generated parameters and enter alternate values for projection image acquisition and reconstruction options. Where the operator selects to override one or more parameters, an update step S750 executes, accepting operator instructions for alternative acquisition or reconstruction parameters and returning to display step S730 with the updated values. Where no further override is needed, a scan enable step S760 then enables the CBCT scan capability using the obtained set of parameters. An acquisition and reconstruction step S770 then performs the required processing for acquisition of a set of 2-D projection images of the volume, and reconstruction of the 3-D volume from the set of 2-D images for display, storage, or transmission.

### Patient-Specific Data and Clinical Parameters

As shown in the FIG. 7 sequence, input parameters provide the basis for recommending a particular set of image capture and reconstruction options. Input parameters for patient imaging, provided as part of accept parameters step S710 in FIG. 7 can include any of the following:

(i) Anatomy to be imaged. The subject or region of interest for a CBCT reconstruction can determine what default conditions apply for initial image capture and subsequent processing.

(ii) Clinical task. The clinical task relates to the particular features of interest from the exam. These can be features of particular bone detail, such a fracture or particular trabecular structure. Soft tissue contrast may be of interest. Alternately, imaging of implants or other hardware can be of interest, such as to evaluate healing, aging, or loosening or degradation, for example.

(iii) Image quality target. Target values for image quality can vary according to the anatomy and clinical task, as outlined previously. There can also be factors such as confidence level that apply for a particular image type.

(iv) Patient descriptors. Patient-specific data that are factors related to image acquisition for the individual patient and can include relative size of the patient or extremity, including relative size of the body part that is to be imaged (small/medium/large or other relative indication), patient condition, patient age, and weight, for example. The individual patient's state of agitation can also be a factor affecting operating options selection, such as suggesting difficulties the particular patient may be having due to pain or due to difficulties with sitting still or remaining in a fixed position for a suitable length of time.

(v) Acquisition speed. The speed of image acquisition can relate to the patient's state of agitation, as just noted, or to urgency or other factors.

(vi) Reconstruction options. Reconstruction options can include overall reconstruction speed, correction for metal content or for patient motion, or factors that relate to selection of a suitable reconstruction protocol.

### Acquisition Parameters

A set of acquisition parameters can be generated automatically from the input parameters obtained in step S710 of the FIG. 7 process. Acquisition parameters define how the scanner operates and define features of the various radiation sources for patient exposure. Among acquisition parameters generated in step S720 of the FIG. 7 sequence can be the following:

(i) Angular range. As was shown in FIG. 1, it may not be feasible or necessary to achieve a full 360-degree rotation of source and detector around the limb of interest. Depending on limb of interest and other input parameters, a suitable angular range can be computed for the scanning operation, providing a full scan or a partial scan between an initial acquisition angle and a final acquisition angle.

(ii) Angular resolution. Depending on the type of exam and needed reconstruction quality, there can be some allowance provided for adjusting the size of the angular increments between captured projection images.

(iii) ROI selection. The selection of an appropriate region of interest (ROI) can be obtained from a scout image, for example. A scout image is acquired separately from the projection images and can be analyzed to detect an ROI using techniques familiar to those skilled in the imaging arts. This selection can be used to designate a suitable combination of one or more x-ray sources, as described previously with reference to FIG. 6.

(iv) X-ray source allocation and conditioning. Along with the number of x-ray sources that are used, acquisition parameters can include data on the number of projections per x-ray source, as well as various technique parameters, including mA/ms per x-ray source, kVp per source, mA/kVp modulation, spectral CT per x-ray source (for example kV switching, filter switching), and filter selection, which can also be varied per x-ray source.

(v) Collimation settings. Where multiple sources are used, collimator settings can differ or can be the same for each source.

(vi) Detector binning mode. Detector binning can be used as a factor in the trade-off between resolution and speed, for example.

(vii) Source/detector trajectory. This can include alterations to the standard gantry arrangement or travel pattern where permitted by the CBCT system.

(viii) Speed. The gantry orbital speed may be adjustable in some embodiments.

(ix) Firing order. Where two or more x-ray sources are selected, the firing order can be adjusted for energizing the sources in a desired sequence.

(x) Number of projections. This value can be computed by the system according to the angular increment and range, as specified above. Alternately, the number of projections can be modified directly by the operator, thereby adjusting the angular increment or range, or both angular increment and range.

(xi) Dual-energy. Dual energy imaging can be specified, with appropriate setup for each cooperating energy channel. Selection of dual-energy imaging can automatically set a number of acquisition variables to default values.

### Reconstruction Parameters

Reconstruction parameters calculated for the input parameters can include any of the following:

(i) Voxel dimensions.

(ii) Reconstruction volume size.

(iii) Reconstruction algorithm used. The system may default to one type of reconstruction algorithm but allow the operator to designate some other type for a particular case. Reconstruction can use an analytical method, such as filtered back projection (FBP) or FDK (Feldkamp, Davis and Kress) reconstruction, or can employ an iterative algebraic or statistical reconstruction technique, for example.

(iv) Reconstruction parameters. The system may generate a default set of reconstruction parameters for the given anatomy or may compute reconstruction parameters appropriate for the exam type. Various types of reconstruction parameter can include variables specifying levels of denoising, regularization, and number of iterations, for example.

Default or computed parameter settings can be displayed to the operator in step S730 of FIG. 7 in order to allow system settings to be reviewed. While the proposed system can determine what parameter settings meet the clinical needs, it is desirable to allow for operator override of computed values, such as by on-screen entry of one or more operator instructions.

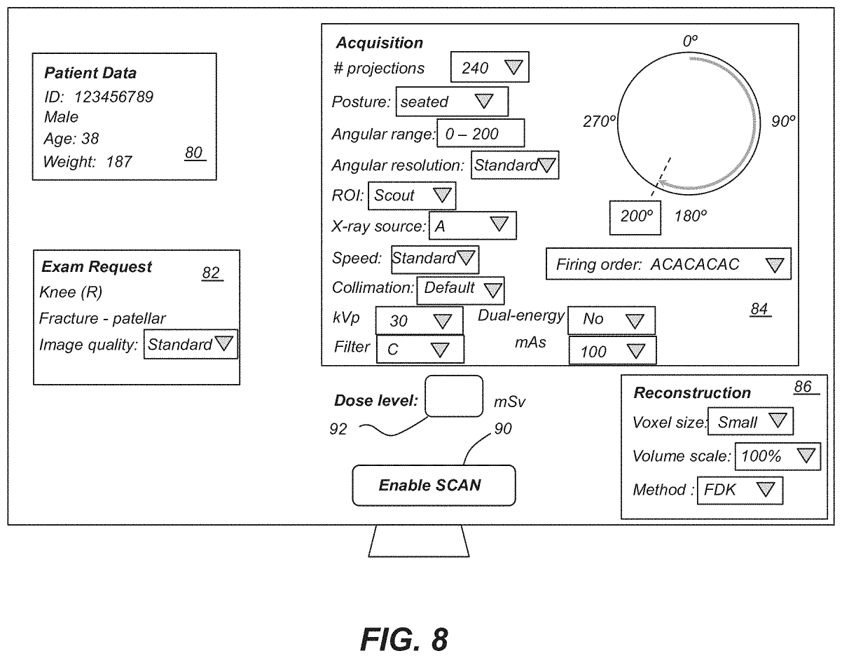

By way of example FIG. 8 shows a display screen that can appear on control panel 124 for showing various parameters related to a CBCT exam for an extremity. A patient data window 80 provides various patient descriptors with identifying data and information for the individual patient. An exam data window 82 includes information on the requested exam, identifying the anatomy of interest, the clinical task, and aspects of the exam such as image quality target. One or more data fields in exam data window 82 may offer options to attending personnel, such as allowing adjustments related to image quality where time would be saved or exposure minimized, for example.

Continuing with the FIG. 8 control interface, an acquisition settings window 84 allows the CBCT operator a number of options for adapting system operation to the clinical need for the specific patient, as described previously. Fields that dictate operational parameters can be initially populated with default values for the requested exam and considering various patient-specific factors, such as age and weight, for example. A number of parameters may allow adjustment, such as allowing menu selections for changing various parameter fields, as shown in FIG. 8. For example, there may be reasons for changing the patient posture from a standard posture typically used for the exam type to some other positioning. For some extremity types, the angular range may allow some adjustment. FIG. 8 shows an example of an interface for angular adjustment that can allow the operator to adjust the range of angles, along with specifying starting and ending angles for the scan, shown in the example as between 0 and 200 degrees. The configuration and use of x-ray sources can be specified, such as selecting one from a number of sources or specifying a combination that employs multiple sources. Depending on the exam type, parameters such as scanning speed, collimation, and angular resolution may be adjustable. A reconstruction options window 86 allows the operator to override default variables for parameters such as voxel dimension, scale factors, and reconstruction method, for example. A scan enable control 90 then allows scanning to be executed according to the operator approved parameters.

A dose indicator 92 shows a computed dose value for the given parameters. This value can be a numeric value, such as in milliSieverts (mSv) or some other indication (low/medium/high) and may be initially obtained using a scout image. According to an embodiment, the dose level is adjustable; the FIG. 8 interface highlights values that can be changed to adjust dose or values that automatically change when the operator adjusts the dose indicator 92 value itself.

Overrides such as those depicted in FIG. 8 can be useful in a number of cases. For example, when a patient appears highly agitated, there may be justification to obtain the needed projection images as quickly as is feasible, even at relatively higher dose than had been computed. Alternately, such a condition could modify the number of projection images acquired. An advisory message can be displayed in cases where operator override adversely impacts dose or requires the patient to assume an uncomfortable position, for example. This can allow the operator to re-consider override of recommended settings in some cases.

Consistent with one embodiment, the present invention utilizes a computer program with stored instructions that perform on image data accessed from an electronic memory. As can be appreciated by those skilled in the image processing arts, a computer program of an embodiment of the present invention can be utilized by a suitable, general-purpose computer system, such as a personal computer or workstation. However, many other types of computer systems can be used to execute the computer program of the present invention, including networked processors. The computer program for performing the method of the present invention may be stored in a computer readable storage medium. This medium may comprise, for example; magnetic storage media such as a magnetic disk such as a hard drive or removable device or magnetic tape; optical storage media such as an optical disc, optical tape, or machine-readable bar code; solid state electronic storage devices such as random-access memory (RAM), or read only memory (ROM); or any other physical device or medium employed to store a computer program. The computer program for performing the method of the present invention may also be stored on computer readable storage medium that is connected to the image processor by way of the internet or other communication medium. Those skilled in the art will readily recognize that the equivalent of such a computer program product may also be constructed in hardware.

It should be noted that the term “memory”, equivalent to “computer-accessible memory” in the context of the present disclosure, can refer to any type of temporary or more enduring data storage workspace used for storing and operating upon image data and accessible to a computer system, including a database. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Displaying an image requires memory storage. Display data, for example, is typically stored in a temporary storage buffer that is directly associated with a display device and is periodically refreshed as needed in order to provide displayed data. This temporary storage buffer can also be considered to be a memory, as the term is used in the present disclosure. Memory is also used as the data workspace for executing and storing intermediate and final results of calculations and other processing. Computer-accessible memory can be volatile, non-volatile, or a hybrid combination of volatile and non-volatile types.

It will be understood that the computer program product of the present invention may make use of various image manipulation algorithms and processes that are well known. It will be further understood that the computer program product embodiment of the present invention may embody algorithms and processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product of the present invention, are not specifically shown or described herein and may be selected from such algorithms, systems, hardware, components and elements known in the art.

The invention has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

1. A method, executed at least in part by a computer, comprising:
a) accepting patient-specific data for imaging a patient extremity on a cone beam computed tomography (CBCT) apparatus;
b) generating and displaying, according to the patient-specific data, a set of acquisition parameters and a dose indication for 2-D projection image capture of the extremity on the CBCT apparatus;
d) updating the generated set of acquisition parameters and dose indication according to one or more operator instructions;
e) acquiring a set of 2-D projection images of a 3-D volume that includes the patient extremity according to the updated set of acquisition parameters;
f) reconstructing the 3-D volume according to the acquired set of 2-D projection images; and
g) displaying, storing, or transmitting at least a portion of the reconstructed 3-D volume.

2. The method of claim 1 further comprising displaying a generated set of reconstruction parameters and updating the reconstruction parameters according to one or more operator-entered instructions.

3. The method of claim 2 wherein the generated set of reconstruction parameters includes a voxel dimension.

4. The method of claim 2 wherein the generated set of reconstruction parameters includes a reconstruction algorithm.

5. The method of claim 1 wherein the CBCT apparatus has two or more x-ray sources and wherein the generated and displayed acquisition parameters specify using at least two of the two or more x-ray sources.

6. The method of claim 5 wherein the operator-entered instruction changes a firing order for the specified two or more x-ray sources.

7. The method of claim 1 wherein the displayed acquisition parameters include at least one parameter taken from the group consisting of an angular range, an angular resolution, a kVp value, and an mAs value.

8. The method of claim 1 further comprising automatically identifying a region of interest and one or more acquisition parameters from a scout image.

9. The method of claim 1 wherein reconstructing the volume uses an iterative reconstruction algorithm.

10. The method of claim 1 wherein reconstructing the volume uses an analytic reconstruction algorithm.

11. The method of claim 1 wherein updating the set of acquisition parameters comprises specifying dual-energy imaging.

12. A method comprising:
a) accepting one or more input parameters for a patient exam of an extremity on a cone beam computed tomography (CBCT) apparatus;
b) generating, according to the accepted input parameters for the patient exam, a set of acquisition parameters for 2-D projection image capture of a region of interest of the extremity using the CBCT apparatus, wherein the acquisition parameters include a combination of two or more x-ray sources;
c) displaying, on a display, the generated set of acquisition parameters;
d) updating at least one of the generated set of acquisition parameters according to an operator instruction;
e) acquiring a set of 2-D projection images of the extremity by scanning the region of interest according to the updated set of acquisition parameters;
f) reconstructing a volume including the region of interest according to the acquired set of 2-D projection images; and
g) rendering at least a portion of the reconstructed volume to the display.

13. The method of claim 12 further comprising storing or transmitting the reconstructed volume.

14. The method of claim 12 further comprising generating and displaying an indicator of patient dose for the extremity exam.

15. The method of claim 12 wherein the set of acquisition parameters includes an adjustable angular range.

16. The method of claim 12 wherein the set of acquisition parameters includes parameters for dual-energy imaging.

17. A cone-beam computed tomography (CBCT) apparatus comprising:
a) a gantry that has a plurality of x-ray sources disposed opposite a digital radiography detector; and
b) a control logic processor in signal communication with the gantry and a display, wherein the processor is configured with programmed instructions to:
(i) accept input parameters for imaging a patient extremity on the CBCT apparatus;
(ii) generate and display a set of acquisition parameters for 2-D projection image capture of the extremity on the CBCT apparatus and a dose indication;
(iii) accept one or more operator instructions to update the generated set of acquisition parameters and dose indication;
(iv) acquire a set of 2-D projection images of a volume according to the updated set of acquisition parameters; and
(v) reconstruct the volume according to the acquired set of 2-D projection images.

18. The apparatus of claim 17 wherein the control logic processor is configured to modify the firing order of two or more of the plurality of x-ray sources.
